(19) United States
(12) Patent Application Publication
(10) Pub. No.: US 2020/0247745 A1
(43) Pub. Date: Aug. 6, 2020

(54) PREPARATION OF 2-CYCLOHEXYLIDEN-2-PHENYL ACETONITRILE AND ODORIFEROUS STRUCTURAL ANALOGS THEREOF

(71) Applicant: Agan Aroma & fine chemicals Ltd., Ashdod (IL)

(72) Inventors: Eyal BEN-ARI, Yavne (IL); Maia AIZENMAN, Rehovot (IL); Sasson SHEMESH, Rehovot (IL); Youlia HAGOOLY, Tel-Aviv (IL)

(73) Assignee: Agan Aroma & fine chemicals Ltd., Ashdod (IL)

(21) Appl. No.: 16/612,776

(22) PCT Filed: May 10, 2018
(86) PCT No.: PCT/IL2018/050515

§ 371 Date: Nov. 12, 2019

Related U.S. Application Data
(60) Provisional application No. 62/505,126, filed on May 12, 2017.

(51) Int. Cl. C07C 253/30; C11B 9/00; C07C 253/34

(52) U.S. Cl. CPC C07C 253/30; C07C 255/34; C07C 253/34; C11B 9/0065

(57) ABSTRACT

A process of preparing 2-cyclohexyliden-2-phenyl acetonitrile or odoriferous structural analogs thereof, represented by Formula I:

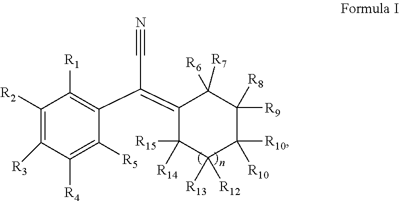

wherein the variables are as defined in the specification, is provided. The process utilizes a phase transfer catalyst, is performed at a temperature lower than 80° C., and provides odoriferous substances at a high yield and purity. Odoriferous substances obtainable by the process, and odor-imparting formulations and articles-of-manufacturing containing same are also provided

### FIELD AND BACKGROUND OF THE INVENTION

The present invention, in some embodiments thereof, relates to odoriferous substances and, more particularly, but not exclusively, to a novel process of preparing 2-cyclohexyliden-2-phenyl acetonitrile or structural analogs thereof, to substances obtainable by the process, and to uses of these substances as odoriferous substances.

2-Cyclohexyliden-2-phenyl acetonitrile, also known and marketed as Peonile®, CAS No. 10461-98-0, is an odoriferous compound featuring floral, geranium, grapefruit, fresh odor. 2-Cyclohexyliden-2-phenyl acetonitrile is a powerful odoriferous compound, is relatively non-volatile (featuring a boiling temperature of about 350 ° C. at 760 mm Hg) and is very stable in almost all media. It has very high substantivity, of 400 hours, which is expressed on both wet and dry substrates. 2-Cyclohexyliden-2-phenyl acetonitrile helps increasing the volume and tenacity in functional perfumes.

2-Cyclohexyliden-2-phenyl acetonitrile is known as highly suitable for use as a perfume (odor-imparting) agent in products such as body care products, including bath/shower gels, hair conditioners, shampoos, liquid soaps, tablet soaps, and talcum powders; perfume products, particularly alcoholic perfumes; cleansing products such as liquid detergents; fabric care products such as fabric softeners; and in lifestyle products, such as pot pourri and incense.

U.S. Pat. No. 6,069,125, to Givaudan Roure SA, discloses the use of 2-cyclohexyliden-2-phenyl acetonitrile as an odoriferous fragrance, and refers to V. J. Harding and W. N. Haworth in J. Chem. Soc. (1910), 486-498 (referred to hereinafter as “Harding”), as describing it as the product of alkaline condensation of phenylacetonitrile and cyclohexanone. According to Harding, phenylacetonitrile was mixed with a solution of sodium in ethyl acetate and after cooling, cyclohexanone was added. The product was heated on a water-bath, cooled, acidified, extracted with ether, worked up and distilled.

Birch and Kon, J. Chem. Soc., Trans., 1923, 123, 2440-2448, and White and Cope, J. Am. Chem. Soc. 65 (1943), 1999-2000, also describe the same synthetic pathway for obtaining 2-cyclohexyliden-2-phenyl acetonitrile. Birch and Kon describe that a pure substance was difficult to obtain. White and Cope report that the product was obtained in 76% yield.

U.S. Pat. No. 7,528,103 discloses benzylic nitrile derivatives featuring linear alkyl groups instead of the cyclohexylidene in 2-Cyclohexyliden-2-phenyl acetonitrile, and their use as perfuming ingredients. U.S. Pat. No. 7,528,103 further discloses a process of preparing such compounds, by reacting a benzylic nitrile compound with an alcohol corresponding to the alkyl group, in the presence of catalytic system comprising a base having a pKa above 13 (e.g., KOH, NaOH or DBU) and a Ruthenium chloride complex, at a temperature above 100° C.

U.S. Pat. No. 7,655,701, to Givaudan, discloses cycloalkylidene(ortho-substituted phenyl)-acetonitriles, and their use as odorants in fragrance applications such as perfumes, household products, laundry product, body care products and cosmetics. The compounds disclosed in this document are prepared by condensation of an ortho-substituted benzyl cyanide and cyclohexanone, in the presence of KOH or sodium methylate as a base, while heating the mixture to temperatures of 80° C. or higher, with concomitant azeotropic distillation. U.S. Patent Application Publication No. 20100021413 describes malodor counteracting compositions which comprise 2-cyclohexyliden-2-phenyl acetonitrile or derivatives thereof. The 2-cyclohexyliden-2-phenyl acetonitrile and its derivatives are prepared, according to this publication, by condensation of a substituted or unsubstituted benzyl cyanide and cyclohexanone, in the presence of KOH, while heating the mixture to temperatures of 120° C. or higher, with concomitant azeotropic distillation.

WO 2013/139766, by Givaudan, discloses compounds comprising a I3-thio carbonyl or nitrile moiety which liberates odor molecules having α,β-unsaturated ketone, aldehyde or nitrile, including 2-Cyclohexyliden-2-phenyl acetonitrile.

WO 2008/063635, WO 2008/152543, WO 2010/132531, WO 2014/189906. WO 2014/189980, WO 2015/051054, WO 2015/051139 and U.S. Patent Application Publication No. 2010/0261629 teach various methodologies for encapsulating or otherwise provide sustained delivery of odor substances, particularly odor substance usable in fabric care products, including 2-Cyclohexyliden-2-phenyl acetonitrile.

SU Patent No. 732250 describes a process in which benzyl cyanide is reacted with a cyclic carbonyl compound in the presence of triethylbenzylammonium chloride as a catalyst, an aqueous solution of sodium hydroxide and an organic solvent. The molar ratio of the carbonyl compound:benzyl cyanide:catalyst is 2.5:1:1, and the reaction time is 6-7 hours.

Additional background art includes U.S. Pat. Nos. 2,762,812 and 3,408,396.

### SUMMARY OF THE INVENTION

According to an aspect of some embodiments of the present invention there is provided a process of preparing a compound of Formula I:

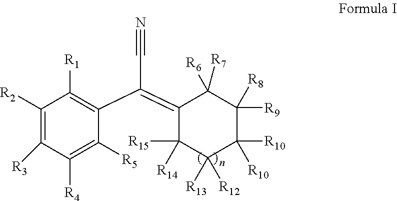

the process comprising:

contacting a mixture of a compound of Formula II:

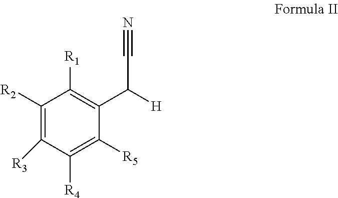

and a compound of Formula III:

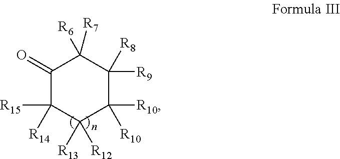

with an alkaline substance and a phase-transfer catalyst, the contacting being at a temperature lower than 80 or lower than 70° C.,

thereby obtaining a reaction mixture comprising the compound of Formula I,

wherein:

n is 0 or 1;

R₁-R₅ are each independently selected from hydrogen, alkyl and alkoxy; and

R₆-R₁₅ are each independently selected from hydrogen and alkyl.

According to some of any of the embodiments described herein, R₁ is selected from hydrogen and alkyl, and R₂-R₅ are each hydrogen.

According to some of any of the embodiments described herein, R₁ is hydrogen.

According to some of any of the embodiments described herein, R₁ is methyl.

According to some of any of the embodiments described herein, each of R₆-R₁₁, R₁₄ and R₁₅, and each of R₁₂ and R₁₃, if present, is hydrogen.

According to some of any of the embodiments described herein, n is 1.

According to some of any of the embodiments described herein, the contacting is for a time period of from 1 hour to 5 hours.

According to some of any of the embodiments described herein, the contacting comprises gradually contacting the mixture with the alkaline substance during a time period of 0.5-2 hours, to thereby obtain the reaction mixture, and heating the reaction mixture at the temperature for an additional time period of from 0.5 to 3 hours.

According to some of any of the embodiments described herein, a mol ratio of the compound of Formula III and the compound of Formula II ranges from 2:1 to 1:2, and according to some embodiments it is 1:1.

According to some of any of the embodiments described herein, a mol ratio of the compound of Formula III and the compound of Formula II is no more than 2:1, or no more than 1.8:1 or no more than 1.7:1, or no more than 1.5:1, or no more than 1.4:1, or no more than 1.3:1 or no more than 1.2:1.

According to some of any of the embodiments described herein, the reaction mixture is devoid of an organic solvent.

According to some of any of the embodiments described herein, the alkaline substance is sodium hydroxide.

According to some of any of the embodiments described herein, the contacting is with an aqueous solution containing the alkaline substance.

According to some of any of the embodiments described herein, a concentration of the alkaline substance in the aqueous solution is from 1 to 90% by weight.

According to some of any of the embodiments described herein, a mol ratio of the alkaline substance and the compound of Formula II is from 10:1 to 1:10.

According to some of any of the embodiments described herein, a mol ratio of the phase transfer catalyst and the compound of Formula II ranges from 1:2000 to 1:1, or from 1:2000 to 1:2, or from 1:2000 to 1:5, or from 1:2000 to 1:10.

According to some of any of the embodiments described herein, the process further comprises, subsequent to the contacting, isolating the compound of Formula I from the reaction mixture, to thereby obtain a reaction product comprising the compound of Formula I.

According to some of any of the embodiments described herein, the reaction product comprises at least 80, or at least 85, or at last 90, weight percents, of the compound of Formula I.

According to some of any of the embodiments described herein, the reaction product is devoid of a substance resulting from a hydrolysis of the compound of Formula II.

According to some of any of the embodiments described herein, the isolating further comprising purifying the reaction product, to thereby obtain an odoriferous substance comprising at least 99% by weight of the compound of Formula I.

According to some of any of the embodiments described herein, the purifying is devoid of chromatography or from any means for separating a substance resulting from a hydrolysis of the compound of Formula II from the compound of Formula I.

According to some of any of the embodiments described herein, a yield of the compound of Formula I is at least 85% relative to the compound of Formula II.

According to an aspect of some embodiments of the present invention there is provided an odoriferous substance comprising the compound of Formula I, obtainable by the process as described herein in any of the respective embodiments and any combination thereof.

According to an aspect of some embodiments of the present invention there is provided an odor-imparting formulation (a fragrance formulation) comprising the odoriferous substance as described herein in any of the respective embodiments and at least one additional odoriferous substance.

According to an aspect of some embodiments of the present invention there is provided an article-of-manufacturing comprising the odoriferous substance or the odor-imparting formulation as described herein in any of the respective embodiments and any combination thereof.

According to an aspect of some embodiments of the present invention there is provided a reaction product comprising a compound of Formula I, as described herein, obtainable by contacting a compound of Formula II, as described herein, and a compound of Formula III, as described herein, in the presence of an alkaline substance, as described herein, the reaction product comprising, prior to isolating and/or purifying the compound of Formula I, at least 80%, or at least 85%, or at least 90%, by weight of the compound of Formula I.

According to some of any of the embodiments described herein, prior to isolating and/or purifying the compound of Formula I, the (crude) reaction product is devoid of a substance resulting from a hydrolysis of the compound of Formula II.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-C present GC chromatograms of samples of a crude product obtained by a process as described in Reference Example 2 (FIG. 1A), Reference Example 3 (FIG. 1B) and Example 1 (FIG. 1C); GC analyses were performed using Agilent 7890A GCw with a Restek RXi-5ms: 30 m×250 micrometer×0.25 micrometer column, operated at an oven temperature of 40° C. for 0 minutes, then 10° C./minute to 240° C. for 1 minute, and then 40° C./minute to 310 for 6 minutes.

Table 1 presents a summary of the reaction conditions and parameters and corresponding yield of the processes described in Reference Examples 1-4 and in Example 1.

### DESCRIPTION OF SPECIFIC EMBODIMENTS OF THE INVENTION

The present invention, in some embodiments thereof, relates to odoriferous substances and, more particularly, but not exclusively, to a novel process of preparing 2-cyclohexyliden-2-phenyl acetonitrile and structural analogs thereof, to substances obtainable by the process, and to uses of these substances as odoriferous substances.

As discussed in the Background section hereinabove, 2-cyclohexyliden-2-phenyl acetonitrile is known as a powerful odoriferous substance featuring floral, geranium, grapefruit, fresh odor, which is widely used in a myriad of fragrance applications. Some structural analogs of 2-cyclohexyliden-2-phenyl acetonitrile have also been described as odoriferous substances. 2-cyclohexyliden-2-phenyl acetonitrile is a synthetic odoriferous substance. As further discussed hereinabove, the preparation of 2-cyclohexyliden-2-phenyl acetonitrile and of structural analogs thereof involves a condensation reaction between a respective phenylacetonitrile (e.g., benzyl cyanide, BnCN) and a respective ketone (e.g., cyclohexanone). This condensation reaction is known in the art to be effected in the presence of a base (an alkaline substance) and optionally a catalyst such as an amphiphilic substance (e.g., a polyethylene glycol) or an organometallic complex. The currently practiced synthetic protocols, however, result in relatively low yield of the product (75-80%). See, for example, U.S. Pat. Nos. 7,655,701 and 7,528,103, U.S. Patent Application Publication No. 20100021413 and Reference Examples 1-4 in the Examples section that follows.

In addition to the relatively low yield, the currently practiced synthetic protocols are further less than optimal, as using relatively expensive and/or hazardous reagents, for example, a large excess of cyclohexanone (e.g., a mol ratio of 1.5:1, relative to BnCN), or a relatively high amount of a catalyst, which triggers either expensive and laborious recycling techniques or means for safely discarding these substances. Further, the currently practiced synthetic protocols require performing the condensation reaction at relatively high temperatures, above 100° C. (e.g., 100-150° C.) and at prolonged reaction times of about 4-7 hours, which are energy consuming. Overall, the currently practiced processes are time-consuming and energy-consuming, utilize excessive amounts of hazardous and/or expensive reagents, and are therefore economically inefficient and environmentally unfriendly.

The present inventors have studied the currently practiced synthetic protocols for preparing 2-Cyclohexyliden-2-phenyl acetonitrile and have uncovered that in addition to the above-mentioned disadvantageous, these processes typically result in a complex crude product that requires laborious and industrially inefficient steps for rendering the product sufficiently purified for being used as an odoriferous substance. For further reference, see the Examples section that follows.

In a search for a more efficient synthetic methodology for preparing the widely used 2-cyclohexyliden-2-phenyl acetonitrile odoriferous substance, and structural analogs thereof, particularly odoriferous structural analogs thereof, the present inventors have designed and successfully practiced a novel synthetic process (method), which is cost-effective, less time- and energy-consuming, avoids the need to recycle reagents, and circumvents the need to use laborious purification procedures. Moreover, the process affords the 2-cyclohexyliden-2-phenyl acetonitrile or its structural analogs in high yield and high purity.

Embodiments of the present invention therefore relate to a process of preparing 2-cyclohexyliden-2-phenyl acetonitrile and structural analogs thereof, which are collectively represented herein by Formula I, to synthetically prepared odoriferous products obtainable by the process and to odor-imparting formulations containing same, and to articles-of-manufacturing comprising these synthetically-prepared products and formulations containing same.

The process of the present embodiments is highly suitable for commercial-scale manufacturing of the odoriferous products as described herein.

By “commercial scale manufacturing” it is meant that a product is synthetically prepared in each batch in an amount that is proportional to a commercial amount. A “commercial amount” can be regarded as an amount of a product that is marketed by a manufacturer per year so as to meet the requirements of a relevant market.

For example, of a commercial amount is 12 tons per year, a commercial scale manufacturing is of batches that produce 100 Kg of the product per month, about 25 Kg of the product per week, and about 5 Kg of a product per day.

In some embodiments, a commercial scale manufacturing process is for producing at least 1 Kg of the odoriferous product in each batch.

In some embodiments, a commercial scale manufacturing process used at least 1 Kg of a compound of Formula II or of a compound of Formula III in each batch.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

### The Odoriferous Substances

The phrase “odoriferous substance”, as used herein and in the art, describes a chemical substance or a mixture of chemical substances featuring an odor which is commonly conceived as pleasant.

According to some of any of the embodiments described herein, the odoriferous substance comprises at least 85%, or at least 86%, or at least 87%, or at least 88%, or at least 89%, or at least 90%, or at least 91% or at least 92%, or at least 93%, or at least 94%, or at least 95%, preferably at least 96%, at least 97%, at least 98%, at least 99%, or more, by weight, of the total weight of the odoriferous substance, the compound 2-Cyclohexyliden-2-phenyl acetonitrile and/or of odoriferous structural analogs thereof, which are collectively represented herein by Formula I:

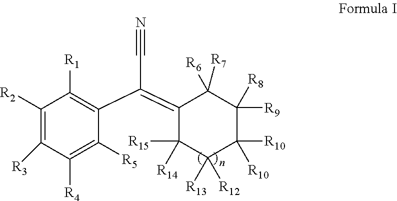

wherein:

n is 0 or 1;

R₁-R₅ are each independently selected from hydrogen, alkyl and alkoxy; and

R₆-R₁₅ are each independently selected from hydrogen and alkyl.

Herein, the term “alkyl” describes a saturated aliphatic hydrocarbon including straight chain and branched chain groups. Preferably, the alkyl group has 1 to 20 carbon atoms. Whenever a numerical range; e.g., “1-20”, is stated herein, it implies that the group, in this case the alkyl group, may contain 1 carbon atom, 2 carbon atoms, 3 carbon atoms, etc., up to and including 20 carbon atoms. More preferably, the alkyl is a medium size alkyl having 1 to 10 carbon atoms. Most preferably, unless otherwise indicated, the alkyl is a lower alkyl having 1 to 4 carbon atoms (C(1-4) alkyl) or even 1 to 3 carbon atoms (C(1-3) alkyl). Exemplary alkyls include methyl, ethyl and propyl, preferably unsubstituted.

Herein, the term “alkoxy” describes an —O-alkyl group, with the alkyl being as described herein.

In some of any of the embodiments described herein, n is 1, and R₁-R₁₅ are each hydrogen, the compound being 2-Cyclohexyliden-2-phenyl acetonitrile.

In some of any of the embodiments described herein, n is 0, and R₁-R₁₅ are each hydrogen, the compound being a cyclopentylidene analog of 2-Cyclohexyliden-2-phenyl acetonitrile, that is, 2-Cyclopentyliden-2-phenyl acetonitrile.

In some of any of the embodiments described herein, n is 0 or 1, and R₁ is alkyl, preferably methyl. In some of these embodiments, R₂-R₅ are each hydrogen.

In some of these embodiments, each of R₆-R₁₁, R₁₄ and R₁₅, and each of R₁₂ and R₁₃, if present, is hydrogen.

In some of these embodiments, R₁ is alkyl, preferably methyl, n is 1, R₂-R₁₅ are each hydrogen, the compound being an ortho-tolyl or o-tolyl analog of 2-Cyclohexyliden-2-phenyl acetonitrile, or 2-Cyclohexyliden-2-o-tolyl acetonitrile

In some of any of the embodiments described herein, each of R₆-R₁₁, R₁₄ and R₁₅, and each of R₁₂ and R₁₃, if present, is hydrogen.

Alternatively, one or more of R₆-R₁₁, R₁₄ and R₁₅, and R₁₂ and R₁₃, if present, is other than hydrogen, and can independently be, for example, alkyl such as methyl, ethyl, propyl, and isopropyl, or alkoxy such as ethoxy and methoxy.

Exemplary compounds of Formula I which are known as usable odoriferous substances include, without limitation, 2-Cyclohexyliden-2-phenyl acetonitrile, 2-cyclohexylidene-2-o-tolyl acetonitrile, 2-cyclopentylidene-2-o-tolyl-acetonitrile, 2-(2-methoxycyclohexylidene)-2-phenyl acetonitrile, and 2-(2-methylcyclohexylidene)-2-o-tolyl acetonitrile.

In the context of the present embodiments, a compound may exhibit one or more chiral centers, each of which exhibiting an R- or an S-configuration and any combination, and compounds according to embodiments of the present invention can have any one of their chiral centers exhibiting an R- or an S-configuration, or can be a racemic mixture comprising both R- and an S-configurations of one or more of their chiral centers.

According to some of any of the embodiments described herein, the compound of Formula I or an odoriferous substance comprising same, as defined herein, is obtainable by a process as described herein.

### The Process

The process, according to the present embodiments, comprises performing a condensation reaction between a compound of Formula II (phenyl acetonitrile or a derivative thereof):

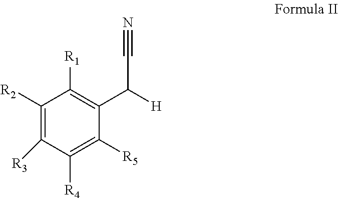

and a compound of Formula III (cyclohexanone, cyclopentanone or a derivative thereof):

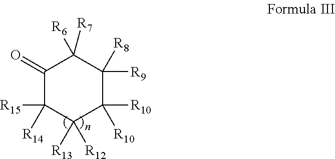

wherein n and R₁-R₁₅ are as described in any one of the respective embodiments.

The phenylacetonitrile or the derivative thereof (a substituted phenylacetonitrile in which one or more of Ri-Rs is other than hydrogen) and the cyclohexanone, cyclopentanone or derivatives thereof (a substituted cyclohexanone or cyclophenatone, in which one or more of R₈-R₁₅ is other than hydrogen) are selected in accordance with the desired structure of the synthesized odoriferous substance.

According to the present embodiments, the condensation reaction is performed in the presence of alkaline substance (a base)) and a phase transfer catalyst (PTC), as defined herein.

In some embodiments, the condensation reaction is effected by contacting a compound of Formula II, a compound of Formula III, an alkaline substance as described herein, and a phase transfer catalyst, as described herein.

In some embodiments, the condensation reaction is effected by contacting a mixture of a compound of Formula II and a compound of Formula III, with an alkaline substance as described herein and a phase transfer catalyst as described herein.

In some of any of the embodiments described herein, the condensation reaction (the contacting) is effected at a temperature of no more than 100° C. In some of any of the embodiments described herein, the condensation reaction (the contacting) is effected at a temperature lower than 100° C., or lower than 90° C., preferably lower than 80° C., or lower than 70° C., more preferably lower than 60° C., for example at a temperature range of from 45° C. to 55° C., or at about 55° C.

In some of any of the embodiments described herein, the condensation reaction (the contacting) is effected at a temperature ranging from room temperature (e.g., 20 or 25° C.) to about 100° C. In some of any of the embodiments described herein, the condensation reaction (the contacting) is effected at a temperature ranging from room temperature to about 70° C., or from room temperature to about 60° C., for example at a temperature range of from about 20° C. to about 60° C., or from about 25° C. to about 60° C., or from about 30° C. to about 60° C., or from about 40° C. to about 60° C. In some embodiments, the reaction (the contacting) is effected at room temperature. In some embodiments, it is effected at a temperature of from about 20° C. to about 30° C.

In some of any of the embodiments described herein, the process comprises contacting a mixture of a compound of Formula II and a compound of Formula III with an alkaline substance as described herein and a phase transfer catalyst as described herein, at a temperature as described herein.

In some of any of the embodiments described herein, the condensation reaction is effected during a time period of less than 7 hours or less than 6 hours, or of about 5 hours, and preferably less, e.g., from about 2 to about 3 hours. Longer and shorter time periods are also contemplated.

In some of any of the embodiments described herein, contacting a mixture of a compound of Formula II and a compound of Formula III with an alkaline substance as described herein and a phase transfer catalyst as described herein is for a time period as described herein.

In some of any of the embodiments described herein, contacting a mixture of a compound of Formula II and a compound of Formula III with an alkaline substance as described herein and a phase transfer catalyst as described herein comprises gradually adding the mixture of a compound of Formula II and a compound of Formula III to a mixture of the alkaline substance and the PTC, to thereby obtain a reaction mixture, while heating the obtained reaction mixture at a temperature as described herein. In some embodiments, the alkaline substance and the PTC as described herein are placed in a reaction container and heated at the indicated temperature, and the mixture of a compound of Formula II and a compound of Formula III is added to the reaction container gradually, while maintaining the temperature of the obtained reaction mixture.

In some embodiments, the mixture of a compound of Formula II and a compound of Formula III is added to the reaction container containing the alkaline substance and the PTC during a time period of from about 1 minute to about 2 hours, or from about 10 minutes to about 2 hours, or from about 30 minutes (0.5 hour) to about 2 hours, or from about 30 minutes to about 90 minutes (e.g., about 1 hour), including any intermediate values and subranges therebetween, to thereby obtain a reaction mixture, and the obtained reaction mixture is heated at a temperature as described herein for an additional time period of from about 0.5 hour to about 3 hours, or from about 1 hour to about 2 hours (e.g., 1.5 hour), including any intermediate values and subranges therebetween.

Thus, according to embodiments of the present invention, the condensation reaction (contacting) is performed at an overall relatively short total time period of 1-5, or 2-3 hours, and at a relatively low temperature, e.g., as described herein in any of the respective embodiments.

According to some of any of the embodiments described herein, the condensation reaction is performed in the presence of a catalytic system which comprises an alkaline substance and a phase transfer catalyst.

The alkaline substance can be any substance that provides a pH higher than 12. Exemplary alkaline substances include, but are not limited to, a hydroxide of an alkali or earth metal such as sodium hydroxide, potassium hydroxide, magnesium hydroxide, lithium hydroxide and like substances, or, alternatively, sodium bicarbonate, sodium carbonate, calcium carbonate, potassium carbonate, potassium bicarbonate, and like substances.

Preferably, the alkaline substance is a hydroxide of an alkali metal, such as sodium hydroxide or potassium hydroxide, which renders the process more cost-effective compared, for example, to carbonate bases.

In some embodiments, the alkaline substance is sodium hydroxide.

In some embodiments, the alkaline substance is used per se, as a solid (e.g., as a powder, granules, pellets or tablets).

In some embodiments, the alkaline substance is used as an alkaline aqueous solution which comprises water and the alkaline substance (a base). Depending on the concentration of the alkaline substance and its solubility in water, the aqueous solution can be such that the alkaline substance is completely dissolved, partially dissolved, dispersed or it can be in a form of a slurry.

The term “aqueous solution” thus encompasses solutions, dispersions, suspensions, and slurries, comprising the alkaline substance and water at various concentrations of the alkaline substance.

In some embodiments, the alkaline aqueous solution features a pH 12 or higher, e.g., of 12-14, or of 13.

The concentration of the alkaline substance in the aqueous solution can range from 0.01 to 99.99%, or from 1 to 99%, by weight, of the total weight of the alkaline aqueous solution, including any intermediate values and subranges therebetween.

In some embodiments, the alkaline aqueous solution is a concentrated solution, and a concentration of the alkaline substance in the aqueous solution is at least 20% by weight, preferably at least 30% by weight, more preferably at least 40%, by weight, of the total weight of the aqueous solution. In some embodiments, a concentration of the alkaline substance in the aqueous solution is from 40 to 50% by weight, and in some embodiments it is about 44% by weight.

In some embodiments, an amount of the alkaline substance or the amount and concentration of the alkaline aqueous solution is such that a mol ratio of the alkaline substance itself (per se) and the compound of Formula III is from10:1 to 1:10, or from 5:1 to 1:5, or from 2:1 to 1:1 or from 1.5:1 to 1:1.5.

In some of any of these embodiments, the mol ratio is about 1:1. In some embodiments, an amount of the alkaline substance or the amount and concentration of the alkaline aqueous solution is such that a mol ratio of the alkaline substance itself (per se) and the compound of Formula II is from10:1 to 1:10, or from 5:1 to 1:5, or from 2:1 to 1:1 or from 1.5:1 to 1:1.5.

In some of any of these embodiments, the mol ratio is about 1:1.

In some of any of the embodiments described herein, the alkaline substance is used as an alkaline aqueous solution of sodium hydroxide, and a concentration of the sodium hydroxide ranges from about 40% to about 50%, by weight, and in some of these embodiments, a mol ratio of sodium hydroxide and a compound of Formula II is about 1:1.

Herein throughout, and in the art, the phrase “phase transfer catalyst”, abbreviated as PTC, describes a substance that promotes a chemical reaction between a reactant in an organic phase and a reactant in an aqueous phase, typically by facilitating the migration of one of the reactants from one phase into another phase where the chemical reaction occurs. Typically, phase transfer catalysts facilitate the migration of ionic substances from an aqueous phase to an organic phase by facilitating the solubilization of the ionic substance in the organic phase, where the other reactant is soluble.

Without being bound by any particular theory, it is assumed that a phase transfer catalyst promotes the reaction between the alkaline substance and the compound of Formula II, which results in formation of an anion that reacts with the compound of Formula III within the condensation reaction.

Exemplary phase transfer catalysts suitable for use in the context of the process of the present embodiments include, but are not limited to, quaternary ammonium salts of the formula:

RaRbRcRdN⁺X⁻

wherein X⁻ is an anionic moiety such as, but not limited to, halo (fluoro, chcloro, bromo, iodo, typically bromo or chloro) nitrate, bisulfate, sulfonate, hydroxy, or BY₄⁻ wherein each Y is independently as defined for X; and Ra, Rb, Rc, Rd are each independently alkyl, cycloalkyl or aryl, as defined herein, or, alternatively, two or more of Ra, Rb, Rc and Rd are linked together to form a heteroalicyclic (saturated or unsaturated) or heteroaryl, as defined herein.

In some of these embodiments, at least one of Ra, Rb, Rc and Rd is an aryl or an alkyl which is a medium to high alkyl, having at least 4 carbon atoms.

Exemplary commercially available quaternary ammonium PTCs include, for example, benzyltrimethylammonium chloride, benzyltriethylammonium chloride, methyltricaprylammonium chloride, methyltributylammonium chloride, and methyltrioctylammonium chloride. Many other quaternary ammonium PCTs are available and all are encompassed by the present embodiments.

In some embodiments, the PTC is tetra-n-butylammonium bromide (TBAB).

In some of any of the embodiments described herein, a mol ratio of the phase transfer catalyst and a compound of Formula II as described herein, ranges from 10:1 to 1:2000, or from 1:1 to 1:2000, or from 1:1 to 1:1500, or from 1:1 to 1:1000, or from 1:1 to 1:500 or from 1:1 to 1:200, or from 1:5 to 1:2000, or from 1:5 to 1:1000, or from 1:5 to 1:500, or from 1:5 to 1:200, or from 1:10 to 1:2000, or from 1:10 to 1:1000, or from 1:10 to 1:500, or from 1:10 to 1:200, or from 1:20 to 1:2000, or from 1:20 to 1:1000, or from 1:20 to 1:500, or from 1:20 to 1:200, or from 1:50 to 1:2000, or from 1:50 to 1:1000, or from 1:50 to 1:500, or from 1:50 to 1:200, including any subranges and intermediate values therebetween, and in some embodiments, it is about 1:100 (such that an amount of the phase transfer catalyst is 1 mol % relative to an amount of a compound of Formula II).

In some of any of the embodiments described herein, a mol ratio of the phase transfer catalyst and a compound of Formula II as described herein is at least 1:5, or at least 1:10, or at least 1:20, such that an amount of the phase transfer catalyst is no more than 20 mol %, or no more than 10 mol %, or no more than 5 mol %, relative to an amount of a compound of

Formula II.

In some of any of the embodiments described herein, a mol ratio of the phase transfer catalyst and a compound of Formula III as described herein, ranges from 10:1 to 1:2000, or from 1:1 to 1:2000, 1:1 to 1:1500, or from 1:1 to 1:1000, or from 1:1 to 1:500, or from 1:1 to 1:200, or from 1:5 to 1:2000, or from 1:5 to 1:1000, or from 1:5 to 1:500, or from 1:5 to 1:200, or from 1:10 to 1:2000, or from 1:10 to 1:1000, or from 1:10 to 1:500, or from 1:10 to 1:200, or from 1:20 to 1:2000, or from 1:20 to 1:1000, or from 1:20 to 1:500, or from 1:20 to 1:200, or from 1:50 to 1:2000, or from 1:50 to 1:1000, or from 1:50 to 1:500, or from 1:50 to 1:200, including any subranges and intermediate values therebetween, and in some embodiments, it is about 1:100 (such that an amount of the phase transfer catalyst is 1 mol % relative to an amount of a compound of Formula III).

In some of any of the embodiments described herein, a mol ratio of the phase transfer catalyst and a compound of Formula III as described herein is at least 1:5, or at least 1:10, or at least 1:20, such that an amount of the phase transfer catalyst is no more than 20 mol %, or no more than 10 mol %, or no more than 5 mol %, relative to an amount of a compound of Formula III.

In some of any of the embodiments, a mol ratio of a compound of Formula III and a compound of Formula II as described herein ranges from 2:1 to 1:2, or from 1.5:1 to 1 to 1:5, or from 1.5:1 to 1:1, or from 1.2:1 to 1:1, and in some embodiments this mol ratio is about 1:1. According to some of any of the embodiments described herein, a mol ratio of the compound of Formula III and the compound of Formula II is no more than 2:1, or no more than 1.8:1 or no more than 1.7:1, or no more than 1.5:1, or no more than 1.4:1, or no more than 1.3:1 or no more than 1.2:1.

As demonstrated in the Examples section that follows, the reaction proceeds to afford the reaction condensation product of Formula I in relatively high yield also when a 1:1 mol ratio of the reactants of Formula II and III is used, contrary to prior art processes in which a compound of Formula III, such as cyclohexanone, is used in a molar excess (e.g., 1.5 mol equivalent relative to a compound of Formula II such as benzyl cyanide). As cyclohexanone and derivatives thereof are relatively expensive reactants, performing the process while using molar excess of cyclohexanone (or a derivative thereof), typically requires recycling the unreacted cyclohexanone is order to render the process cost-effective. The process described herein therefore circumvents the need to perform such re-cycling and is in any way cost-effective by avoiding the need to use excessive amounts of a compound of Formula III as described herein.

In some of any of the embodiments described herein, the condensation reaction (contacting) is effected without using an organic solvent as the reaction medium and/or without using any other organic substance other than the compounds of Formulae II and III.

In some of any of the embodiments described herein, the reaction mixture (obtainable upon contacting the compounds of Formulae II and III, the alkaline substance and the PTC) is devoid of an organic solvent and/or any other organic substance other than the compounds of Formulae II and III.

By “devoid of” it is meant that an amount of an organic solvent or any other organic substance and other than the compounds of Formulae II and III is no more than 2%, or no more than 1%, or no more than 0.5%, or no more than 0.1%, or no more than 0.5%, or no more than 0.1%, or no more than 0.05%, or no more than 0.01%, by weight, and can be even less or null.

In the context of these embodiments, an organic solvent or and/or without using any other organic substance other than the compounds of Formulae II and III encompasses one or more of saturated and unsaturated aliphatic (including alicyclic) hydrocarbons, aromatic hydrocarbons, saturated and unsaturated alicyclic hydrocarbons, saturated and unsaturated halogenated hydrocarbons, aliphatic alcohols, ethers, esters and ketones, which is not a compound of Formula II or III as described herein.

The newly designed process as described herein is therefore further advantageously devoid of organic solvents, which are environmentally unfriendly and commonly hazardous, and require excessive purification procedures and/or recycling and/or waste disposal procedures.

In some of any of the embodiments described herein, condensation reaction (contacting) is performed without performing an azeotropic distillation concomitant with or subsequent to the contacting.

Herein and in the art, an “azeotropic distillation” describes a distillation process in which water is removed from the reaction mixture, typically as a volatile mixture with an organic substance or organic solvent.

In some of any of the embodiments described herein, the process further comprises, following contacting the reaction components for effecting the condensation reaction and obtaining a reaction mixture comprising the compound of Formula I, work-up procedures, for isolating the compound of Formula I from the reaction mixture, to thereby obtain a crude reaction product, as defined hereinbelow.

Isolating the compound of Formula I from the reaction mixture can be performed using means known in the art, including phase separation, washing, distillation and/or chromatographic purification. In some embodiments, isolating the compound of Formula I is devoid of chromatographic purification.

By “chromatographic purification” it is meant a purification technique in which a crude reaction product is eluted through a stationary phase, using a solvent system as a mobile phase. In some embodiments, a chromatographic purification technique is such that utilizes silica (e.g., silica gel) as the stationary phase, and an organic medium is which a compound of Formula I is dissolvable as the mobile phase.

In some embodiments, isolating the compound of Formula I comprises removing the aqueous solution from the reaction mixture obtainable by the contacting described herein, to thereby obtain an organic phase comprising the compound of Formula I.

In some embodiments, the organic phase is contacted with an acidic aqueous solution (e.g., sulfuric acid solution or any other aqueous acidic solution that can neutralize remaining traces of the alkaline substance and/or PTC).

In some embodiments, following the addition of an acidic aqueous solution, an organic solvent is added, and phase separation is effected, that is, the aqueous solution is removed, to thereby obtain an organic phase comprising the compound of Formula I and the organic solvent.

The organic solvent is preferably a solvent in which the compounds of Formulae I, II and III are dissolvable and which can be readily separated from the compound of Formula I.

In some embodiments, the organic solvent is cyclohexane, although other solvents (e.g., toluene, or any other hydrocarbon solvent) are also contemplated. In some embodiments, the organic solvent has a boiling temperature lower from the boiling temperature of a compound of Formula I by at least 50° C., or at least 100° C., at atmospheric pressure (760 mm Hg).

In some embodiments, the organic phase comprising the compound of Formula I and the organic solvent is contacted with an aqueous buffer solution, and the aqueous buffer solution is thereafter removed, to afford again an organic phase comprising the compound of Formula I and the organic solvent.

The aqueous buffer solution preferably has a pH of from 6 to 8, more preferably of from 6.5 to 7.5, more preferably of about 7. An exemplary buffer solution is an aqueous solution of a sodium citrate, for example, sodium citrate dihydrate, although any other buffer solutions are contemplated.

In some embodiments, the process proceeds by removing the organic solvent from an organic phase comprising the compound of Formula I and the organic solvent, to thereby obtain a crude reaction product comprising the compound of Formula I. In some embodiments, removal of the organic solvent is effected by evaporation, optionally at a reduced pressure.

Herein, the phrase “crude reaction product” describes a product obtainable in a chemical process, upon performing a chemical reaction (e.g., a condensation reaction as described herein) and subjecting the obtained reaction mixture to work-up procedures as described herein to thereby isolate the reaction product (a compound of Formula I) from the reaction mixture, but before purification procedures such as chromatographic purification, distillation and/or crystallization.

According to some embodiments of the present invention, the crude reaction product obtainable upon isolating the compound of Formula I as described herein, is devoid of, as defined herein, a substance resulting from hydrolysis of a compound of Formula II.

As shown in the Examples section that follows, substances resulting from hydrolysis of a compound of Formula II cannot be separated from a corresponding compound of Formula I (formed by reacting the compound of Formula II and a compound of Formula III) by distillation and either lead to a final product which does not feature a desirable purity or require additional purification steps (in addition to distillation) for separating it, for example, chromatography.

According to some embodiments of the present invention, the crude reaction product comprises at least 80%, or at least 85%, or at least 86%, or at least 87%, or at least 88%, or at least 89%, or at least 90%, or at least 91%, or at least 92%, or more, of a compound of Formula I.

In some embodiments, isolating the compound of Formula I from the reaction mixture comprises subjecting a crude reaction product as described herein to purification.

In some embodiments, the purification comprises distillation and/or crystallization.

In some embodiments, the purification is devoid of chromatographic purification.

In some embodiments, the purification step does not involve separating a product of the hydrolysis of a compound of Formula II.

In some embodiments, the purification consists of distillation and/or crystallization, and in some embodiments, the purification consists of distillation, as described herein.

In some embodiments, subsequent to isolating a compound of Formula I from the reaction mixture (e.g., subsequent to removing the organic solvent from an organic phase comprising same as described herein), distillation is effected, to further purify the obtained odoriferous substance.

In some embodiments, the distillation is effected at a reduced pressure and at a temperature that preferably does not exceed 200° C. or does not exceed 150° C. or does not exceed 140° C., or does not exceed 130° C., or does not exceed 125° C., or does not exceed 120° C.

Determining a reduced pressure and a corresponding temperature at which a compound of Formula I is distilled off is well within the knowledge of those skilled in the art.

In some embodiments, the distillation is effected at a reduced pressure lower than 100 mmHg, or lower than 10 mmHg, or lower than 5 mmHg, for example, at 1 mm Hg.

As further discussed hereinabove, the process as described herein affords a compound of Formula I at a quantitative yield higher than 80%, higher than 85%, higher than 88%, and even higher (e.g., 90%).

By “quantitative yield” it is meant herein the number of moles of a compound of Formula I compared to the number of moles of a compound of Formula II used in the process.

According to some of any of the embodiments described herein, a process as described herein is characterized by at least one of the following:
(i) A reaction temperature lower than 80° C., or lower than 70° C., more preferably lower than 60° C., for example at a temperature range of from 45° C. to 55° C., or at about 55° C.; and/or
(ii) An overall reaction time (a time period of said contacting) of less than 6 hours, or less than 5 hours, or of 1-5 hours or of 2-3 hours; and/or
(iii) A mol ratio of the compound of Formula III and the compound of Formula II is no more than 2:1, or no more than 1.8:1 or no more than 1.7:1, or no more than 1.5:1, or no more than 1.4:1, or no more than 1.3:1 or no more than 1.2:1; and/or
(iv) A mol ratio of the phase transfer catalyst and a compound of Formula II as described herein is at least 1:5, or at least 1:10, or at least 1:20, such that an amount of the phase transfer catalyst is no more than 20 mol %, or no more than 10 mol %, or no more than 5 mol %, relative to an amount of a compound of Formula II; and/or
(v) A mol ratio of the phase transfer catalyst and a compound of Formula III as described herein is at least 1:5, or at least 1:10, or at least 1:20, such that an amount of the phase transfer catalyst is no more than 20 mol %, or no more than 10 mol %, or no more than 5 mol %, relative to an amount of a compound of Formula III; and/or
(vi) A process devoid of an organic solvent, as described herein.

In some of any of the embodiments described herein, the process is characterized by one, two, three, four, five or all of the above parameters.

As discussed herein, each of the above parameters, let alone a combination of some or all of these parameters, renders the process advantageous to other processed for preparing the odoriferous products described herein.

Each of the above parameters independently, let alone a combination of some or all of these parameters, renders the process advantageous for preparing or manufacturing the odoriferous product in a commercial scale, as described herein in any of the respective embodiments.

### The Reaction Product

As discussed herein, one of the advantages of the process according to the present embodiments is the absence of substances obtained upon hydrolysis of a compound of Formula II as described herein.

According to some embodiments of the present invention there is provided a reaction product comprising a compound of Formula I, as described herein in any of the respective embodiments, obtainable by contacting a compound of Formula II, as described herein in any of the respective embodiments, and a compound of Formula III, as described herein in any of the respective embodiments, in the presence of an alkaline substance, as described herein.

The reaction product can be a crude reaction product as described herein, obtainable upon contacting the compounds of Formulae II and III the reaction product comprising, and isolating the compound of Formula I from the reaction mixture, and prior to purifying the compound of Formula I.

The reaction product comprises at least 85%, or at least 86%, or at least 87%, or at least 88%, or at least 89%, or at least 90%, or at least 91% or even 92%, by weight of the compound of Formula I of the total weight of the reaction product.

In some embodiments, the reaction product, prior to isolating and/or purifying the compound of Formula I (e.g., a crude reaction product as described herein), is devoid of a substance resulting from a hydrolysis of the compound of Formula II, as defined herein. According to some of these embodiments, the compound of Formula I is 2-cyclohexyliden-2-phenyl acetonitrile.

### Applications

As described herein, compounds of Formula I as described herein include odoriferous substances. Such odoriferous substances, obtainable by a process as described herein, can be advantageously incorporated an odor-imparting (fragrance) formulations and/or in articles-of-manufacturing where including such an odoriferous substance is beneficial.

According to an aspect of some embodiments of the present invention, there is provided an article-of-manufacturing comprising odoriferous substance as described herein.

In some embodiments, the articles-of-manufacturing include products to which the addition of an odor-imparting agent is beneficial.

In some embodiments, the articles of manufacturing include body care products, including bath/shower gels, hair conditioners, shampoos, liquid soaps, tablet soaps, cosmetic products and talcum powders; perfume products, particularly alcoholic perfumes; cleansing products or compositions such as liquid detergents; fabric care products such as fabric softeners; and in lifestyle products, such as pot pourri and incense.

Non-limiting examples of such article-of-manufacturing include, baby care, beauty care, fabric and home care, family care, feminine care, health care, snack and/or beverage products, and, more specifically, but without limitation, fine fragrance products or formulations (e.g. perfumes, colognes, eau de toilettes, after-shave lotions, pre-shave, face waters, tonics, and other fragrance-containing compositions for application directly to the skin), diapers, bibs, wipes; products for and/or methods relating to treating hair (human, dog, and/or cat), including, bleaching, coloring, dyeing, conditioning, shampooing, styling formulations or products; deodorants and antiperspirants, personal cleansing, cosmetics and skin care products or formulations, including creams, lotions, and other topically applied products, and shaving products; products for and/or methods relating to treating fabrics, hard surfaces and any other surfaces in the area of fabric and home care, including air care, car care, dishwashing, fabric conditioning (including softening), laundry detergent, laundry and rinse additive and/or care, hard surface cleaning and/or treatment, and other cleaning compositions; products and/or methods relating to bath tissue, facial tissue, paper handkerchiefs, and/or paper towels; tampons, feminine napkins; products and/or methods relating to oral care including toothpastes, tooth gels, tooth rinses, denture adhesives, tooth whitening.

As used herein, the term “cleansing composition” includes washing agents, especially cleaning detergents, liquid, gel or paste-form all-purpose washing agents, liquid fine-fabric detergents, hand dishwashing agents or light duty dishwashing agents, machine dishwashing agents, including the various tablet, granular, liquid and rinse-aid types, cleaning and disinfecting agents, including antibacterial hand-wash types, cleaning bars, mouthwashes, denture cleaners, dentifrice, car or carpet shampoos, bathroom cleaners, hair shampoos and hair-rinses, shower gels and foam baths and metal cleaners, as well as cleaning auxiliaries such as bleach additives and “stain-stick” or pre-treat types, substrate-laden products such as dryer added sheets, dry and wetted wipes and pads, nonwoven substrates, and sponges, as well as sprays and mists.

As used herein, the term “fabric care composition” includes, unless otherwise indicated, fabric softening compositions, fabric enhancing compositions, fabric freshening compositions and combinations thereof.

The odoriferous substance of the present embodiments can be used in combination with one or more other odor-imparting agents (fragrances).

In some embodiments, there is provided a fragrance formulation, comprising the odoriferous substance of the present embodiments, optionally in combination with one or more other odor-imparting agents (fragrances), and optionally an acceptable carrier (e.g., alcoholic or water-containing carrier), which can be provided as a fragrance concentrate of as a fragrance formulation which can be incorporated an article-of-manufacturing as described herein.

The odoriferous substance can be employed in widely varying amounts, depending upon the specific application and on the nature and quantity of other odorant ingredients, if present. The proportion is typically from 0.001 to 20 weight percents of the total weight of the article-of-manufacturing or formulation containing same, but can also be up to 50 weight percents. The odoriferous substance of the present embodiments may be employed simply by directly mixing it, or a fragrance formulation containing same, with the article-of-manufacturing to which it is applied. Optionally, the odoriferous substance or a fragrance formulation containing same may be entrapped or embedded in a delivery system such as, for example, polymers, capsules, microcapsules and nanocapsules, liposomes, film formers, absorbents such as carbon or Zeolites, cyclic oligosaccharides, and mixtures thereof, or may be chemically bonded to substrates, which are adapted to release the odoriferous substance(s) upon application of an external stimulus such as light, enzyme, or the like, and then applied to the article-of-manufacturing.

Embodiments of the present invention thus further encompass methods of manufacturing articles-of-manufacturing as described herein, which comprise incorporating an odoriferous substance or a fragrance formulation containing same in the article-of-manufacturing, typically using conventional techniques and methods. Through the addition of the odoriferous substance of the present embodiments, the odor notes of the article-of-manufacturing are be improved, enhanced or modified.

It is expected that during the life of a patent maturing from this application many relevant article-of-manufacturing to which an odoriferous substance is beneficially added will be developed and the scope of the term “article-of-manufacturing” is intended to include all such new technologies a priori.

It is expected that during the life of a patent maturing from this application many relevant phase transfer catalysts will be developed and the scope of the term “phase transfer catalyst” is intended to include all such new technologies a priori.

As used herein the term “about” refers to ±10% or ±5%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

The term “cycloalkyl” describes an all-carbon monocyclic or fused ring (i.e., rings which share an adjacent pair of carbon atoms) group where one or more of the rings does not have a completely conjugated pi-electron system.

The term “aryl” describes an all-carbon monocyclic or fused-ring polycyclic (i.e., rings which share adjacent pairs of carbon atoms) groups having a completely conjugated pi-electron system.

The term “heteroaryl” describes a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furane, thiophene, imidazole, oxazole, thiazole, pyrazole, pyridine, pyrimidine, quinoline, isoquinoline and purine. The heteroaryl group may be substituted or unsubstituted.

The term “heteroalicyclic” describes a monocyclic or fused ring group having in the ring(s) one or more atoms such as nitrogen, oxygen and sulfur. The rings may also have one or more double bonds. However, the rings do not have a completely conjugated pi-electron system. Representative examples are piperidine, piperazine, tetrahydrofurane, tetrahydropyrane, morpholino and the like.

The term “halide” and “halo” describes fluorine, chlorine, bromine or iodine.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

### Reference Examples 1-4

In an attempt to perform an alkaline condensation between phenyl acetonotrile and cyclohexanone, to thereby produce 2-Cyclohexyliden-2-phenyl acetonitrile (Peonile®), procedures described in the art were used as a starting point, and various modifications of these procedures were performed in order to render the synthesis process more efficient and to improve the purity and yield of the final product.

During these studies, it was found that while practicing synthetic protocols described in the prior art, the reaction mixture yields, a crude reaction product which includes, in addition to the 2-Cyclohexyliden-2-phenyl acetonitrile product, unreacted starting materials and some other side-products, an impurity which cannot be separated from the 2-Cyclohexyliden-2-phenyl acetonitrile product by distillation, and require removal by other purification methodologies in addition to distillation, for example, by silica chromatography.

It was further found that such an impurity is obtained in higher amounts at longer reaction times, yet, reducing the reaction time results in a lower yield of the product.

It was further found that while higher yields are obtained when higher temperatures and/or larger amount of cyclohexanone are employed, such conditions still result in a final yield that is lower than 80%, and in substantial amount of the above-mentioned impurity, thus requiring silica chromatography, or any other additional purification method (in addition to distillation) for obtaining a desirably pure compound.

Reference Examples 1-4 below describe representative examples of synthetic procedures performed based on prior art procedures for preparing 2-Cyclohexyliden-2-phenyl acetonitrile.

In all of the experiments conducted, GC analyses were performed during various stages of the reaction and work up steps, using Agilent 7890A GC with a Restek RXi-5ms: 30 m×250 micrometer×0.25 micrometer column, operated at an oven temperature as follows: 40° C. for 0 minutes, then 10° C./minute to 240° C. for 1 minute; then 40° C./minute to 310° C. for 6 minutes. At this set-up, the retention time (R.T.) of 2-Cyclohexyliden-2-phenyl acetonitrile is typically about 19.4 minutes; of benzyl cyanide is about 10.3 minutes; and of cyclohexanone is about 6.1 minutes. Notable impurities appear at 18.7 minutes (for an isomer of 2-cyclohexyliden-2-phenyl acetonitrile-1-cyclohexeny-1-phenylacetonitril)), at 14-15 minutes (assumed to be a product of the hydrolysis of benzyl cyanide), and at 16.06 minutes and 16.3 minutes). Of these, the impurity appearing at a retention time of 14-15 minutes could not be separated from 2-cyclohexyliden-2-phenyl acetonitrile during distillation and it was found that an effective separation of this impurity can be effected by silica chromatography of the obtained crude product prior to its distillation.

In all of the procedures described in Examples 1-4 below, the final crude product, obtained after working-up the reaction mixture, and optionally upon subjecting the crude product to silica chromatography, if performed), can be subjected to distillation, typically at a pressure of 1 mm Hg, at which 2-Cyclohexyliden-2-phenyl acetonitrile distills off at 118-119° C.

### Reference Example 1

The alkaline condensation of phenyl acetonitrile and cyclohexanone was performed using KOH as a base, in accordance with the procedure described, for example, in U.S. Patent Application Publication No. 20100021413, and depicted in Scheme 1 below.

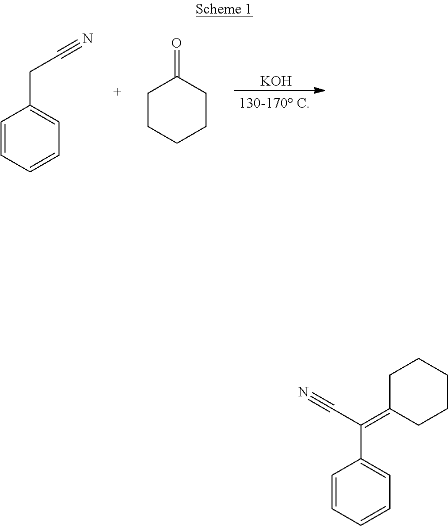

Cyclohexanone (22.5 grams, 1.4 mol equivalents, 0.23 mol), KOH (1.12 grams, 0.125 mol equivalents, 0.02 mol), and benzyl cyanide (phenyl acetonitrile; 18.7 grams, 1 mol equivalents, 0.16 mol) were placed in a 250 ml 3-neck round bottom flask, equipped with a thermometer, a stirrer, and a Dean-Stark apparatus. The reaction mixture was heated to 130° C. After 1 hour, azeotropic distillation started. The reaction mixture temperature was then raised to 170° C., for one hour, and then cooled to 60° C.

To the reaction mixture was added 25 ml toluene and the organic phase was washed with water at 60° C. After phase separation the organic phase was washed with 10% sodium carbonate solution. All aqueous phases were extracted with toluene and then the organic phases were combined, washed three times with 25 ml water, dried with MgSO₄ and toluene was evaporated. GC qualitative analysis of the obtained crude product showed it includes 70.3% of 2-cyclohexyliden-2-phenyl acetonitrile, which was translated to 68% yield in a quantitative analysis; 5.6% benzyl cyanide, which indicates that 94.4% of benzyl cyanide were consumed;

and numerous impurities, including the impurity at R.T. of 14-15 minutes at 0.5%.

### Reference Example 2

The alkaline condensation of phenyl acetonitrile and cyclohexanone was performed using sodium methoxide in ethanol as an alkaline solution, in accordance with the procedure described in WO 2008/0200554, and depicted in Scheme 2 below.

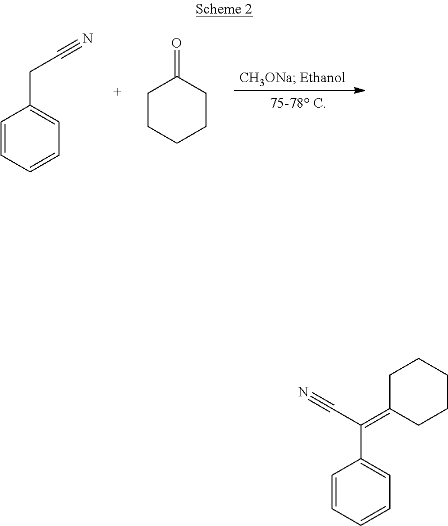

Sodium methoxide (5.4 grams, 0.1 mol, 1 mol equivalent, used as 18 grams of a 30% sodium methoxide solution) was dissolved in ethanol (70 ml), benzyl cyanide (phenyl acetonitrile; 11.7 grams, 1 mol equivalents, 0.1 mol) was added thereto dropwise, during 5 minutes, and the reaction mixture was stirred at room temperature for 15 minutes. Cyclohexanone (9.8 grams, 1 mol equivalents, 0.1 mol) was then added dropwise during 50 minutes, and the reaction mixture was stirred at room temperature for 30 minutes, and then heated at 75-78° C. for 4.5 hours.

To the obtained reaction mixture was added toluene (50 ml) and water (40 ml). The water phase was extracted three times with toluene. All organic phases were combined, washed with saturated sodium carbonate solution and with saturated NaC1 solution, toluene was evaporated. GC qualitative analysis of the obtained crude reaction showed it includes about 3.7% unreacted benzyl cyanide, 5.2% unreacted cyclohexanone, about 70.5% 2-cyclohexyliden-2-phenyl acetonitrile, and numerous impurities, including about 0.7% of the impurity at a retention time of 14-15, and about 4% of the isomer of 2-cyclohexyliden-2-phenyl acetonitrile. According to quantitative analysis of the crude product the obtained yield was 60%.

### Reference Example 3

In an attempt to improve the alkaline condensation as practiced in Reference Example 1, the reaction was performed using KOH as a base, in presence of poly(ethylene glycol) (PEG) and of toluene as a solvent.

Toluene (300 ml) and KOH (11.2 grams; 0.2 mol; 0.2 mol equivalent) were placed in a 1000 ml 4-neck round bottom flask equipped with a thermometer, a stirrer and a Dean-Stark apparatus and the mixture was heated to 110° C. (within about 30 minutes). A mixture of benzyl cyanide (117 grams; 1 mol; 1 mol equivalent) and cyclohexanone (100 grams, 1.02 mol, 1 mol equivalent) was then added while heating at 110° C. was maintained, during 4.5 hours. Azeotrope distillation was observed during 4 hours and 55 ml of azeotrope were obtained.

Thereafter water was added and the mixture was stirred for 30 minutes at 60° C. Phases were separated, the water phase was extracted with toluene and combined with the main organic phase, and toluene was evaporated. GC qualitative analysis of the obtained crude reaction showed it includes 6.3% of unreacted benzyl cyanide and 8.6% of unreacted; about 7.6% of the impurity at R.T. 14-15;, and about 0.65% of the isomer. Overall yield of the product was about 70%, according to quantitative analysis of the crude reaction product.

### Reference Example 4

2-Cyclohexyliden-2-phenyl acetonitrile was prepared while further modifying the procedure described in Reference Example 3. KOH was replaced by potassium carbonate, as depicted in Scheme 3 below, and a portion of the benzyl cyanide was added dropwise.

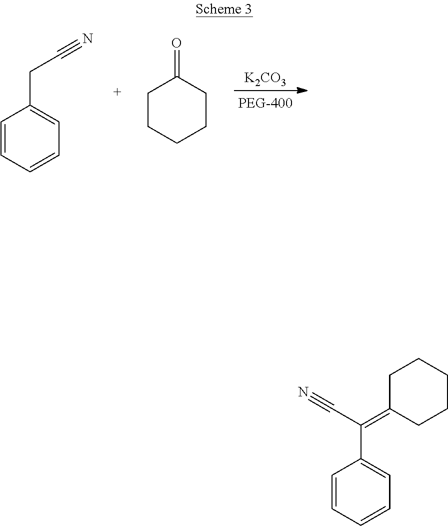

### Materials

Equipment: 1L 4-neck round bottom flask, thermometer, dropping funnel, Dean-Stark apparatus with CaCl₂ tube and condenser.

Reagents: Benzyl cyanide, cyclohexanone, potassium carbonate (K₂CO₃), polyethylene glycol 400 (PEG-400). For work-up process: distilled water (DW), toluene.

### Synthetic Protocol

Cyclohexanone (30 grams, 1.5 mol equivalents, 0.3 mol), K₂CO₃ (13.8 grams, 0. 5 mol equivalents, 0.1 mol), PEG-400 (40 grams, 0.5 molequivalent, 0.1 mol), and 30% of the total amount of benzyl cyanide (7.3 grams, 0.3 mol equivalents, 0.066 mol) were placed in a 250 ml 4-neck round bottom flask, equipped with a thermometer, a dropping funnel, a Dean-Stark apparatus with CaC12 tube and a condenser.

The remaining amount of benzyl cyanide (164 grams, 0.7 mol equivalents, 0.133 mol) was added to the reaction mixture during 1 hour and 15 minutes while heating the reaction mixture at 120-130° C.

The reaction mixture was thereafter heated for 4 hours and 40 minutes at 120-130° C., during which an azeotropic distillation (T=95° C. in vapor) of cyclohexanone/water (38.4%/61.6%) (lower layer-water, upper layer-cyclohexanone) occurred. The azeotropic distillation was finished after about 4 hours.

At the end of reaction, a sample of the reaction mixture was analyzed by GC, indicating that it contained BnCN 4.3%, Cyclohexanone 19.5%, and 2-cyclohexyliden-2-phenyl acetonitrile 68%.

The reaction mixture was cooled to 60° C. and 69.4 grams of toluene and 100 grams of distilled water (DW) were added. The obtained mixture was stirred for 30 minutes at 60° C. Phases were separated, and additional 70 grams water was added to the organic phase and the obtained mixture was stirred for 30 minutes at 60° C. The organic phase was acidified with 10% acetic acid until pH of 6-7 was obtained, and 55 grams water was added and the obtained mixture was stirred for 30 minutes at 60° C.

The organic phases were combined and GC analysis of the obtained organic phase showed it includes 29% 2-cyclohexyliden-2-phenyl acetonitrile, 3.3% and 0.4% of cyclohexanone dimers, 0.3% of the impurity at R.T. of 14-15 minutes and 3% of the isomer of 2-cyclohexyliden-2-phenylacetonitril. Yield of 2-cyclohexyliden-2-phenylacetonitril according to quantitative GC analysis was 78%.

The combined organic phase was purified by filtration though a silica gel column. In brief, 65 grams of silica gel (60A, 0.063-0.200mm) were transferred into a filter AMMA 50 micron, washed with 75 grams of toluene, and thereafter 780 grams of the combined organic phases were passed through the column with elution rate of 280 ml/hour. At the end of filtration the silica gel column was washed with 40 grams toluene.

The purified organic phase was evaporated (under reduced pressure of 30 mbar, and at a bath temperature of 60° C.). 400 grams of a distillate were obtained, and 445 grams of a crude product.

The crude product contained 70% 2-Cyclohexyliden-2-phenyl acetonitrile, as determined by GC chromatography (quantitative analysis).

The crude product was further purified by distillation as described hereinabove (1 mmHg, 118-119° C.), to afford 240 grams of 2-cyclohexyliden-2-phenyl acetonitrile (62% yield), at a purity higher than 99%, as determined by GC analysis.

### Example 1

The present inventors have designed and practiced a novel methodology for performing the condensation reaction between cyclohexanone and benzyl cyanide in order to obtain 2-Cyclohexyliden-2-phenyl acetonitrile. In this methodology, the reaction is performed at a lower temperature, shorter reaction time, using smaller amount of cyclohexanone, yet results at higher yields and purity level of the product. Importantly, the obtained crude reaction product does not include the impurity appearing at retention time 14-15, which is obtained while using prior art methodologies (see, Reference Examples 2-4, for example), and hence laborious separation of this impurity (by e.g., chromatography as described for Reference Example 4) is avoided.

An exemplary process of preparing 2-Cyclohexyliden-2-phenyl acetonitrile according this methodology is schematically illustrated in Scheme 4 below:

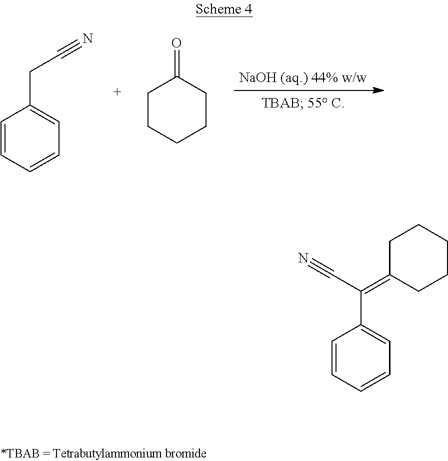

An aqueous solution of NaOH (44%; 295 grams), was added into a 1 L flask, under N₂ flow, 10.47 grams of TBAB were added thereto and the mixture was heated to 45° C.

A mixture of cyclohexanone (98%; 328.4 grams) and benzyl Cyanide (387.64 grams) was then added to the heated reaction flask, during 1 hour, while maintaining the reaction temperature between 50 to 55° C., and the obtained reaction mixture was stirred at 55° C. for additional 1.5 hours.

At the end of reaction, a sample of the reaction mixture was analyzed by GC, indicating that it contained BnCN<5%, Cyclohexanone<3%, 2-cyclohexyliden-2-phenyl acetonitrile≥90%, dimers of cyclohexanone<0.2, and the impurity at R.T. of 14-15 minutes>0.01%.

Stirring was then stopped, phase separation occurred, and the alkaline aqueous phase was removed. The remaining organic phase was cooled to 45° C., and then an aqueous solution of sulfuric acid solution (1.5%; 150 grams) was added, and the obtained mixture was stirred for 20 minutes at 55° C., during which 120 grams of cyclohexane were added.

Stirring was then stopped, the reaction mixture was heated at to 50° C. and phase separation occurred after about 20 minutes. The acidic aqueous phase was removed, and the remaining organic phase was cooled to 45° C., water (295 grams) was added, and the obtained mixture was stirred for 20 minutes at 45° C. Stirring was then stopped, and phase separation occurred after about 30 minutes. The aqueous phase (pH 6.5-7.5) was removed, water (200 grams) was added and the mixture was stirred for 20 minutes at 50° C. Stirring was then stopped, and phase separation occurred after about 15 minutes. The aqueous phase was removed, and cyclohexane was evaporated, to thereby afford 660 grams of a crude reaction product containing 86-92% 2-cyclohexyliden-2-phenyl acetonitrile, at 90% yield, as determined by GC.

The crude reaction product was subjected to distillation, as described hereinabove (1 mmHg, 118-119° C.) for Reference Examples 4, yielding 514 grams (72% final yield), of 2-cyclohexyliden-2-phenyl acetonitrile, having a purity higher than 99%, as determined by GC.

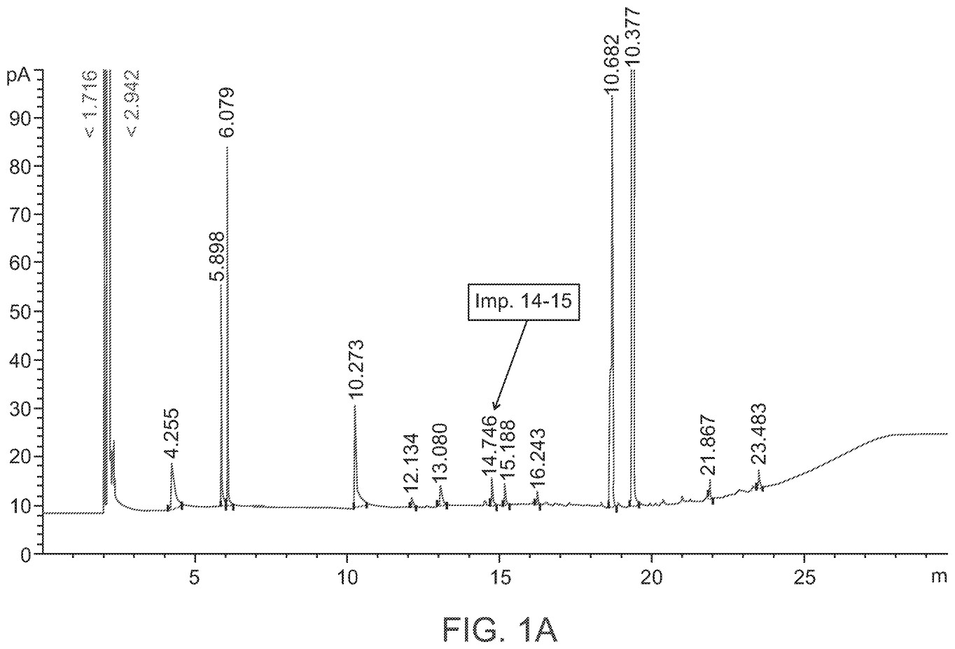

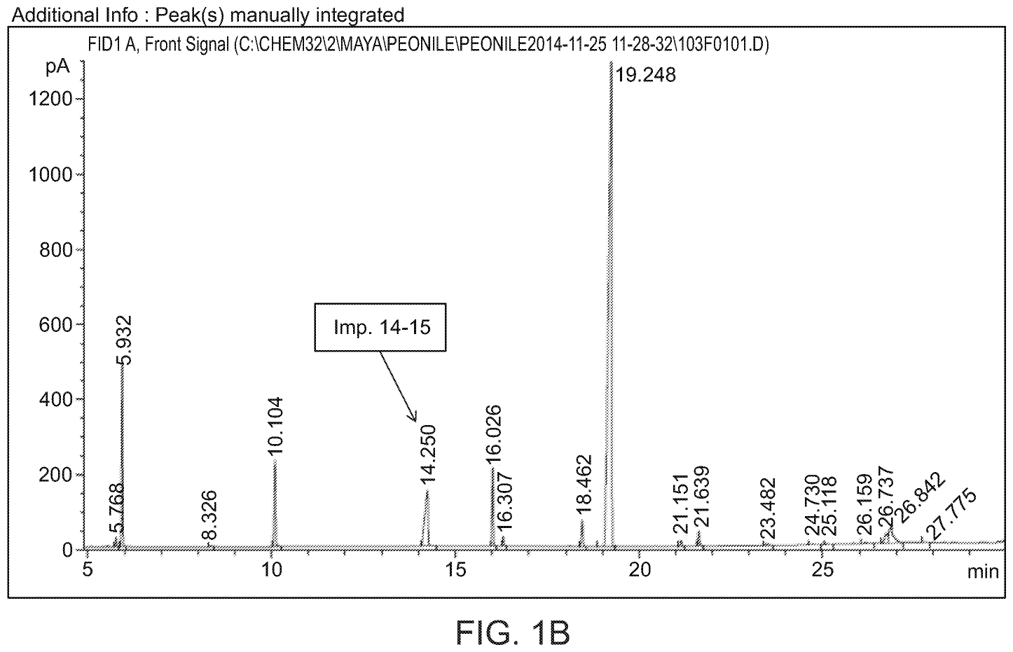

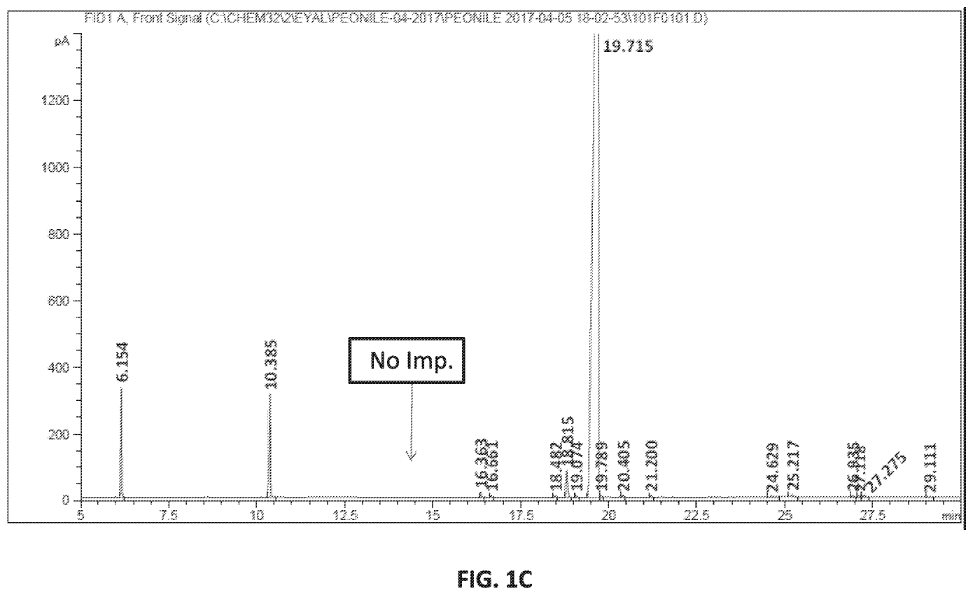

FIGS. 1A-C present GC chromatograms of the crude reaction product, obtained following heating and after work-up of procedures as exemplified in Reference Example 2 (FIG. 1A), Reference Example 3 (FIG. 1B), and in Example 1 herein (FIG. 1C). As seen therein, the impurity at R.T. of 14-15 minutes appears in the crude reaction products obtained in Reference Examples 2 and 3, but not in the crude reaction product obtained in Example 1.

Table 1 summarizes the reaction parameters and conditions and obtained yield of the process as exemplified in Example 1 compared to Reference Examples 1-4.

Table 1 further demonstrates that improvement obtained by a process according to exemplary embodiments of the present invention (Example 1), showing that a higher yield of the purified product is obtained, at lower reaction temperature, shorter reaction time, and without a need to perform chromatography.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

TABLE 1

| | Cyclo | Benzyl | | | Total | Imp at | | Work up |
|---|---|---|---|---|---|---|---|---|
| Example | -hexanone | Cyanide | Base | Temp. | Reaction | 14.2 (% | | (before |
| No. | (mol eq.) | (mol eq.) | (mol eq.) | (° C.) | Time (h) | area in GC) | Yield | distillation) |
| Ref. 1 | 1.4 | 1 | KOH | 130-170 | 4 | Not | 75% | |
| | | | (0.125) | | | determined | | |
| Ref. 2 | 1 | 1 | NaOMe | 25-78 | 5.5 | 0.70% | 60% | washing |
| | | | (1) | | | | | |
| Ref. 3 | 1 | 1 | KOH/PEG | 100-110 | 5 | 7.60% | 73.6% | washing |
| | | | (0.2/0.5) | | | | | |
| Ref. 4 | 1.5 | 1 | K₂CO₃/PEG | 120-130 | about 6 | 0.27% | 78% | Washing + |
| | | | (0.5/0.5) | | | | | silica |
| 1 | 1 | 1 | NaOH 44% | 50-55 | 2.5 | 0-0.007% | 86-90% | washing |
| | | | (0.5-1) | | | | | |

1. A process of preparing a compound of Formula I: 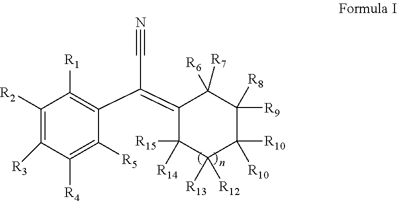
the process comprising:
contacting a mixture of a compound of Formula II: 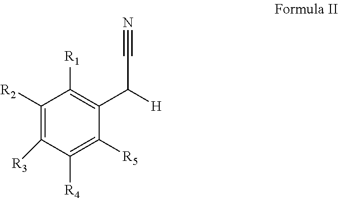
and a compound of Formula III: 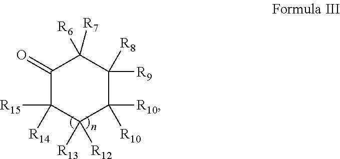
with an alkaline substance and a phase-transfer catalyst, said contacting being at a temperature lower than 80 or lower than 70° C.,
thereby obtaining a reaction mixture comprising said compound of Formula I,
wherein:
n is 0 or 1;
R₁-R₅ are each independently selected from hydrogen, alkyl and alkoxy; and
R₆-R₁₅ are each independently selected from hydrogen and alkyl.

2. The process of claim 1, wherein said contacting is for a time period of from 1 hour to 5 hours.

3. The process of claim 2, wherein said contacting comprises gradually contacting said mixture with said alkaline substance during a time period of 0.5-2 hours, to thereby obtain said reaction mixture, and heating said reaction mixture at said temperature for an additional time period of from 0.5 to 3 hours.

4. The process of claim 1, wherein a mol ratio of said compound of Formula III and said compound of Formula II ranges from 2:1 to 1:2.

5. The process of claim 1, wherein a mol ratio of said compound of Formula III and said compound of Formula II is no more than 2:1.

6. The process of claim 4, wherein said ratio is 1:1.

7. The process of claim 1, wherein said reaction mixture is devoid of an organic solvent.

8. The process of claim 1, wherein said alkaline substance is sodium hydroxide.

9. The process of claim 1, wherein said contacting is with an aqueous solution containing said alkaline substance.

10. The process of claim 9, wherein a concentration of said alkaline substance in said aqueous solution is from 1 to 90% by weight.

11. The process of claim 1, wherein a mol ratio of said alkaline substance and said compound of Formula II is from 10:1 to 1:10.

12. The process of claim 1, wherein a mol ratio of said phase transfer catalyst and said compound of Formula II is ranges from 1:2000 to 1:1.

13. The process of claim 1, wherein a mol ratio of said phase transfer catalyst and said compound of Formula II is at least 1:5.

14. The process of claim 1, wherein a mol ratio of said phase transfer catalyst and said compound of Formula III is at least 1:5.

15. The process of claim 1, further comprising, subsequent to said contacting, isolating the compound of Formula I from said reaction mixture, to thereby obtain a reaction product comprising said compound of Formula I.

16. The process of claim 15, wherein said reaction product comprises at least 80, or at least 85, or at last 90, weight percents, of the compound of Formula I.

17. The process of claim 15, wherein said reaction product is devoid of a substance resulting from a hydrolysis of said compound of Formula II.

18. The process of claim 15, wherein said isolating further comprises purifying said reaction product, to thereby obtain an odoriferous substance comprising at least 99% by weight of the compound of Formula I.

19. The process of claim 18, wherein said purifying is devoid of chromatography.

20. The process of claim 1, wherein R₁ is selected from hydrogen and alkyl, and R₂-R₅ are each hydrogen.

21. The process of claim 1 wherein R₁ is hydrogen or methyl.

22. The process of claim 21, wherein each of R₆-R₁₁, R₁₄ and R₁₅, and each of R₁₂ and R₁₃, if present, is hydrogen.

23. The process of claim 1, wherein each of R₆-R₁₁, R₁₄ and R₁₅, and each of R₁₂ and R₁₃, if present, is hydrogen.

24. The process of claim 1, wherein n is 1.

25. The process of claim 1, wherein a yield of the compound of Formula I is at least 85% relative to the compound of Formula II.

26. An odoriferous substance comprising the compound of Formula I, obtainable by the process of claim 1.

27. An odor-imparting formulation (a fragrance formulation) comprising the odoriferous substance of claim 26 and at least one additional odoriferous substance.

28. An article-of-manufacturing comprising the odoriferous substance of claim 26.

29. A reaction product comprising a compound of Formula I, obtainable by contacting a compound of Formula II and a compound of Formula III in the presence of an alkaline substance, the reaction product comprising, prior to isolating and/or purifying the compound of Formula I, at least 80%, or at least 85%, or at least 90%, by weight of the compound of Formula I.

30. The reaction product of claim 29, wherein, prior to isolating and/or purifying the compound of Formula I, the reaction product is devoid of a substance resulting from a hydrolysis of said compound of Formula II.

31. An article-of-manufacturing comprising the odor-imparting formulation of claim 27.
